(19) United States
(12) Patent Application Publication
(10) Pub. No.: US 2013/0158563 A1
(43) Pub. Date: Jun. 20, 2013

(54) Article and Method for Assisting Cesarean Section Delivery

(71) Applicant: Winfield Scott Anderson, JR., Palm Beach Gardens, FL (US)

(72) Inventor: Diana Madden Adams, Great Falls, VA (US)

(73) Assignee: Winfield Scott Anderson, JR., Palm Beach Gardens, FL (US)

(21) Appl. No.: 13/720,472

(22) Filed: Dec. 19, 2012

Related U.S. Application Data
(60) Provisional application No. 61/578,001, filed on Dec. 20, 2011.

(51) Int. Cl. A61B 17/42

(52) U.S. Cl. CPC A61B 17/42
USPC 606/122

(57) ABSTRACT

In order to assist in a cesarean section delivery of a human fetus, an elongated, flexible member having a pocket centrally located is used by the surgeon by placing it under the head of the fetus. The ends of the planar member remain outside of uterus and are pulled in order to help the surgeon guide and dislodge the head of the fetus and to position the fetus so that the head can be flexed through the uterine incision and the fetus delivered.

### RELATED APPLICATIONS AND PRIORITY

The present application claims priority to U.S. Provisional Application No. 61/578,001, filed Dec. 20, 2011, the entirety of which is hereby incorporated by reference.

### FIELD OF THE INVENTION

This present application relates to cesarean section delivery and, more particularly, to an article and a method for assisting in cesarean section delivery.

### BACKGROUND

Often times, a cesarean section is performed by a physician when a traditional vaginal delivery would put the baby's or the mother's health or life at risk. However, occasionally, a cesarean section is performed at the request of the mother even when a traditional vaginal delivery would not put the baby's or mother's health at risk. Typically, a cesarean section entails making an incision in the abdomen and uterine walls of a pregnant woman (female human being) and the removal of the fetus through the incision. The incision is referred to as a uterine incision. The uterine incision varies depending on the location of a child in the uterus.

Typically, the head of a fetus is pointed towards the feet of the woman and the feet of the fetus are pointed towards the head of the woman. To deliver the fetus through the incision, the obstetrician may place a hand under the head of the fetus and pull the head of the fetus towards the head of the woman so as to dislodge the head of the fetus from the vaginal area of the woman. After dislodging the head of the fetus, the obstetrician then lifts and guides the head of the fetus out through the incision. Such a delivery is referred to as a “head-first delivery”. Sometimes in cesarean section deliveries, the fetus is delivered feet first through the incision.

It is important in any cesarean section delivery that the obstetrician not flex his wrist against the incision or the uterine area of the woman because, if the obstetrician uses the incision or uterine area of the women as a fulcrum, it can damage the woman. A problem occurs when the fetal head is impacted in the woman's vaginal area because the obstetrician must exert a great deal of force in order to dislodge the fetal head. It can be very difficult for the obstetrician to employ enough strength to dislodge the head of the fetus.

To solve this problem in the past, forceps and vacuums have been suggested. The use of forceps and vacuums are not optimal and, generally, it is preferred that the strength and dexterity of the obstetrician be primarily relied on in order to deliver the fetus through the incision during a cesarean section delivery.

### SUMMARY OF THE INVENTION

An article and accompanying methods for assisting with cesarean section delivery are disclosed. The article and accompanying methods may provide assistance for the delivery of a fetus through a uterine incision during a cesarean section and also to decrease the time and trauma to both the mother and child. The article may be particularly helpful when the fetal head of the fetus is impacted in the vaginal and pelvic area of the woman or during situations where traditional cesarean section procedures may potentially cause injury to the mother or the fetus. As a result, the article and accompanying methods may provide significant assistance to the obstetrician during the delivery of the fetus. In one embodiment, the article may provide added lifting strength to assist the obstetrician or the person who is delivering the fetus through the uterine incision to deliver the fetus quickly and with minimal trauma to the mother and the child.

As noted herein, the present application relates to both an article and method which can be used during delivery of the fetus by cesarean section. Suitably, the article and method provide for a traumatic adjunct to the delivering obstetrician's hand, which provides added lift strength, and, if needed, added strength for dislodging the fetal head and elevating it to a level of the uterine incision inside the uterus. From that point, the head of the fetus can be flexed and delivered in the usual manner.

The article of the present application may be an elongated, flexible, planar member having a centrally located pocket with an opening in the pocket for the obstetrician to place a hand into the pocket. The hand of the obstetrician, once in the pocket, may be used to insert and guide the central portion of the planar member under the head of the fetus. The end portions of the planar member may remain outside the body of the woman. The end portions of the planar member may act as hand-holds for the obstetrician's other hand or act as a helper or helpers. These other ends may then be used to lift the fetal head and assist the obstetrician during delivery. Thus, the obstetrician need not rely on a single hand, which is cradling the head of the fetus, in order to provide the needed strength for dislodging an impacted head and for lifting the impacted head through the uterine incision.

The planar member may gently cradle the fetal head, like a hammock, to dislodge the fetal head during delivery. The central portion may expand under the fetal head when it is slid into place by the obstetrician's advancing fingertips.

When the planar member is lifted by its two ends, considerable upward lifting power may be added to the obstetrician's min. The planar member can be used to elevate the fetal head upward and out of the vaginal and pelvis to the level of the uterine incision. Then the head of the fetus may be flexed and delivered in the usual fashion through the uterine incision. The head of the fetus may be in constant contact with and guided mainly by the efforts of the obstetrician's hand. In this particular sense, the method of the present application may not deviate from current practices for delivery by cesarean section. The present application, however, may provide an adjunct where added lifting power is needed at the critical moment during delivery. If and when the head of the fetus is lifted without difficulty, the planar member would not be in the way, take up added space, or interfere with the obstetrician's routine practice.

In one embodiment, the planar member may have a pocket which allows the obstetrician to slip the planar member onto a hand and to fan out the center portion of the planar member underneath the impacted fetal head in the pelvis in such a way that when the two ends of the planar member are pulled upward, it would break the suction to dislodge and lift the head just enough to facilitate usual delivery. This would decrease the time of delivery and trauma to both the fetus and the mother, which is typically associated with these difficult, abdominal deliveries.

In one embodiment, the article of the present application may include an elongated, flexible planar member, and a pocket centrally located on the planar member. The pocket may be adapted to fit a hand, and the pocket may have an opening configured for insertion of the hand into the pocket. In one embodiment, the center portion of the planar member may be preferably wider than the end portions of the planar member and it is in this wide center portion that the pocket may be located.

In one embodiment, preferably, the planar member may have two sets of pleats, which may be longitudinally oriented on the planar member and may be positioned on either side of the pocket. These pleats may demark the wide center portion of the planar member from the narrow end portions of the planar member. In one embodiment, the planar member may measure about one to three feet in length, however, other lengths may be utilized based on desired specifications or uses. The wide center portion of the planar member may, in one embodiment, be preferably about one foot in width, while the end portions of the planar member may be about a half a foot in width.

In another embodiment, the planar member may be made up of fabric that can be sterilized. Ideally, the fabric may be a synthetic fabric, which can be sterilized and reused multiple times. In one embodiment, suitable fabrics may include reinforced nylon, silicone, a non-latex rubber, PVC polymer, and any type of strong, soft, supple, and/or synthetic cloth or mesh material. Alternatively, in one embodiment, the article may be made for a one time use, and can be made from cotton or a mixture of materials that are initially sterile.

Preferably, in one embodiment, the pocket of the article may be oriented transverse to the planar member's long axis and the opening in the pocket may also be transverse to the long axis of the planar member, however, other orientations of the pocket and its opening are contemplated. The purpose of the pocket may be to facilitate the obstetrician with inserting and guiding the center portion of the planar member under the head of the fetus, such as during a cesarean section procedure.

In another embodiment, a method for cesarean section delivery is disclosed. The method may include inserting a center portion of an elongated, flexible planar member through a uterine incision where both end portions of the planar member may remain outside the uterine incision. The method may also include placing the center portion of the planar member under the head of a fetus. Furthermore, the method may include pulling on the end portions of the planar member to move the head of the fetus and to facilitate delivery of the fetus through the uterine incision.

As noted herein, there are situations where the obstetrician or other qualified individual will not necessarily know prior to placing his or her hand on the head of the fetus whether he or she will need assistance in delivering the fetus. As a result, yet another embodiment of the present application may be provided. The method may include inserting a center portion of an elongated, flexible planar member through a uterine incision where both end portions of the planar member remain outside of the uterine incision. Additionally, the method may include placing the center portion of the planar member under the head of a fetus. Furthermore, the method may include delivering the fetus through the uterine incision where the center portion of the planar member is under the head of the fetus.

In such a method, the planar member may be present so that, if it is needed, it can be used by the obstetrician or the obstetrician's helper, but does not necessarily need to have its ends pulled should the obstetrician not need additional lifting.

In the method of the present application, it may be preferred that the planar member has a pocket positioned in the central portion of the planar member. Additionally, the method may include the additional step of having the obstetrician place his hand into the pocket through an opening in the pocket so as to allow the obstetrician to guide the center portion of the planar member through the incision and under the head of the fetus.

Additionally, in one embodiment, the method may include preferably having the hand of the obstetrician guide the head of the fetus throughout the delivery process, while a lift force is applied to the end portions of the planar member. Additionally, it may be preferred that the pulling is stopped once the head of the fetus is aligned vertically with the uterine incision. Then, the hand of the obstetrician may be used to guide the head of the fetus up and through the uterine incision.

In yet another embodiment, the fetus may be delivered head first, however, the method and article of the present application may also be used for a feet-first delivery in order to provide additional lift to the bottom or “breech” buttocks of the fetus. This may be performed in the rare case of a vaginal breech delivery where, indeed, this part of the fetus may be impacted in the maternal pelvis instead of the head being impacted. In the feet-first delivery scenario, the center portion may be placed under the buttocks of the fetus.

These and other aspects of the present application may be more fully understood by reference to one or more of the following drawings.

### BRIEF DESCRIPTION OF THE DRAWINGS

FIG. 1 illustrates the article for assisting a cesarean section delivery according to an embodiment of the present disclosure.

FIG. 2 illustrates a surgeon's hands in the pocket of the article of FIG. 1 according to an embodiment of the present disclosure.

FIG. 3 illustrates an impacted fetal head in the uterus of a pregnant woman.

FIG. 4 illustrates inserting and guiding the article of FIG. 1 under the head of the fetus according to an embodiment of the present disclosure.

FIG. 5 illustrates the center portion of the article of FIG. 1 spread out underneath the head of the fetus and the direction of lift on the head of the fetus to dislodge it from its impacted position according to an embodiment of the present disclosure.

FIG. 6 illustrates that the head of the fetus dislodged and elevated to the proper level for delivery through the uterine incision.

FIG. 7 illustrates a method for assisting a cesarean section delivery according to an embodiment of the invention.

FIG. 8 illustrates an article for assisting a cesarean section delivery including a suction device according to an embodiment of the present disclosure.

### DETAILED DESCRIPTION OF THE INVENTION

An article 10 and accompanying methods for assisting a cesarean section delivery are disclosed. Referring to the Figures, and, in particular, FIGS. 1-6, the article 10 may include an elongated, flexible, planar member 12 that has a centrally located pocket 24 with an opening 26 in the pocket 24 for an obstetrician or other qualified individual to place a hand 30 into the pocket 24. Once in the pocket 24, the hand 30 of the obstetrician, may be utilized to insert the central portion 14 of the planar member 12 through the uterine incision 42 of the woman 40. The central portion 14 of the planar member 12 may then be guided under the head 66 or any other desired body part of the fetus 64. The end portions 16 and 18 of the planar member 12 may be configured to remain outside the body of the woman 40 during the procedure. Notably, the end portions 16 and 18 of the planar member 12 may be grasped or otherwise held by the obstetrician or by an assistant to provide additional lift force to assist in delivering the fetus 64. Specifically, the end portions 16 and 18 may be pulled in a desired direction to lift the fetal head 66 of the fetus 64 and to assist the obstetrician during delivery. As a result, the obstetrician does not need to rely on using only the obstetrician's single hand 30 that is under the head 66 of the fetus 64 in order to provide the necessary strength and lift force for dislodging the impacted head 66 and for lifting the impacted head 66 out of the woman 40 via the uterine incision 42.

Referring more specifically to FIG. 1, FIG. 1 illustrates article 10 as having an elongated, flexible planar member 12. In one embodiment, the planar member 12 may be comprised of fabric that can be sterilized. For example, the fabric of the planar member 12 may be a synthetic fabric, which can be sterilized and reused on multiple occasions. In one embodiment, suitable fabrics for the planar member 12 may include, but is not limited to, reinforced nylon, silicone, a non-latex rubber, PVC polymer, and/or any type of strong, soft, supple, synthetic cloth or mesh material. In one embodiment, the article 10 may be made for a one time use, and can be made from cotton or a mixture of materials that are configured to be initially sterile. In one embodiment, the planar member 12 may measure approximately one to three feet in length, however, other lengths may be utilized based on the desired specifications or uses of the article 10. Furthermore, the planar member 12 may include a central portion 14 and two long, narrow lifting end portions 16 and 18.

In addition to the planar member 12, the article 10 may be configured to include one or more pleats 20 and 22 that may be configured to allow the central portion 14 and the planar member 12 to stretch when necessary. The pleats 20 and 22 may be utilized to indicate the transition between the end portions 16 and 18 and center portion 14, as shown in FIG. 1. In one embodiment, the pleats 20 and 22 may be longitudinally oriented on the planar member 12 and may be positioned on either side of the pocket 24. Notably, the pleats 20 and 22 may be configured to be oriented in other directions as well. In one embodiment, the wide center portion 14 of the planar member may, in one embodiment, be preferably about one foot in width, while the end portions 16 and 18 of the planar member 12 may be about a half a foot in width. The center portion 14 may include the pocket 24, which may include an opening 26. The opening 26 of the pocket 24 may be configured to accommodate the hand 30 of the obstetrician or a hand of an assistant that is assisting with the cesarean section delivery procedure. In one embodiment, the pocket 24 of the article 10 may be oriented transverse to the planar member's 12 long axis, and the opening 26 in the pocket 24 may also be configured to be transverse to the long axis of the planar member 12, however, other orientations of the pocket and its opening are also contemplated. In one embodiment, additional pockets 24 may be configured to be positioned on the planar member 12. In one embodiment, the article 10 may include a leading edge 28, which may be configured to be positioned under the head 66 of the fetus 64 when the article 10 is positioned into the uterine incision 42 of the woman 40 or at other desire times.

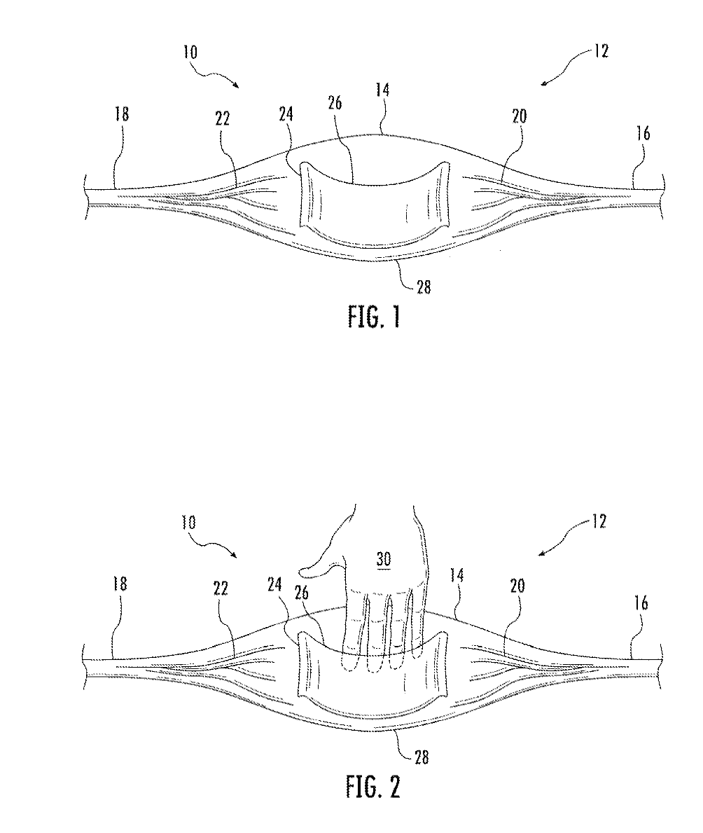

FIG. 2 illustrates article 10 with the surgeon's hand 30 inserted into the pocket 24 of the article 10. The pocket 24 may be configured to allow the surgeon's hand 30 to control the insertion and the location of article 10, and, specifically, to position the center portion 14 of the article 10 under the fetal head 66 of the fetus 64. The fingertips of surgeon's hand 30 may be utilized to guide the leading edge 28 of the article 10 under the head 66 of the fetus 64.

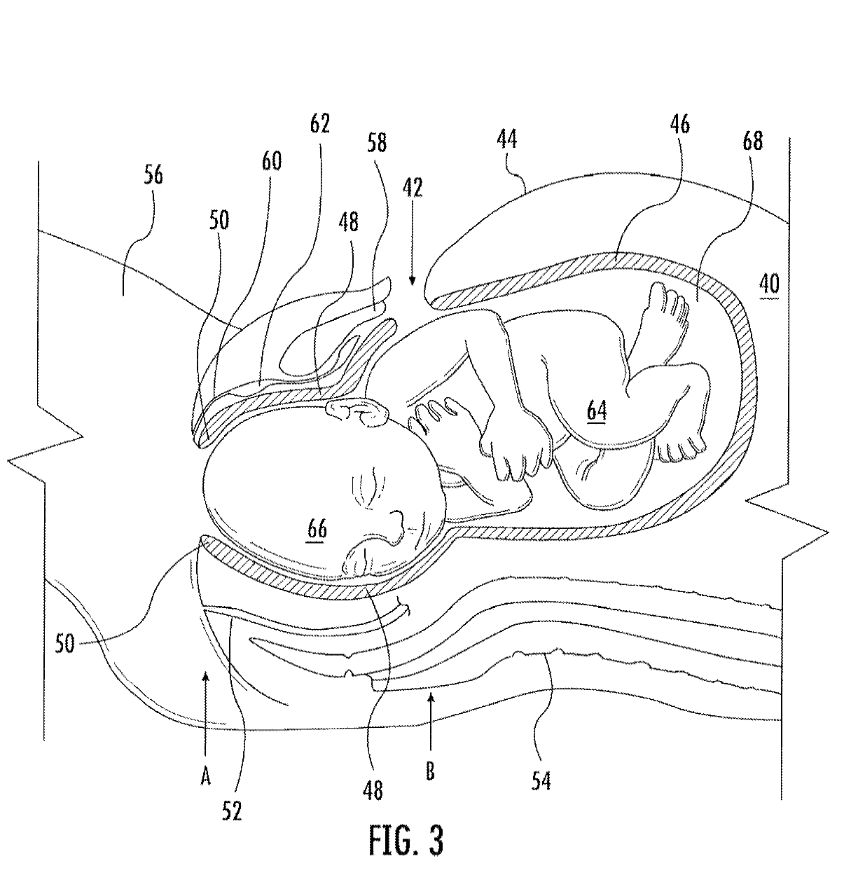

FIG. 3 illustrates an impacted fetal head 66 of the fetus 64 in the pregnant woman 40. The woman 40 undergoing the cesarean section procedure may have a uterine incision 42, an abdominal wall 44, and a uterus wall 46. The uterus wall 46 is made up of the cervix area 48 and the vaginal area 50. The rectum 52, spine 54, leg 56, pubic bone 58, urethra 60, and bladder 62 of the woman 40 are also illustrated for the purposes of illustrating the orientation of the fetus 64 and the fetus head 66 in the uterus 68. As shown in FIG. 3, Location A illustrates the location at which the impacted head 66 of the fetus 64 may rest in the uterus 68 of the woman 40. Location B may be the location at which the fetal head needs to be raised upward towards the head of woman 40 in order to allow flexion and delivery of the fetus 64 through uterine incision 42. Thus, it can be seen that the obstetrician may need to move the fetal head 66 of the fetus 64 from Location A to Location B in order to deliver fetus 64 through uterine incision 42 of the woman 40.

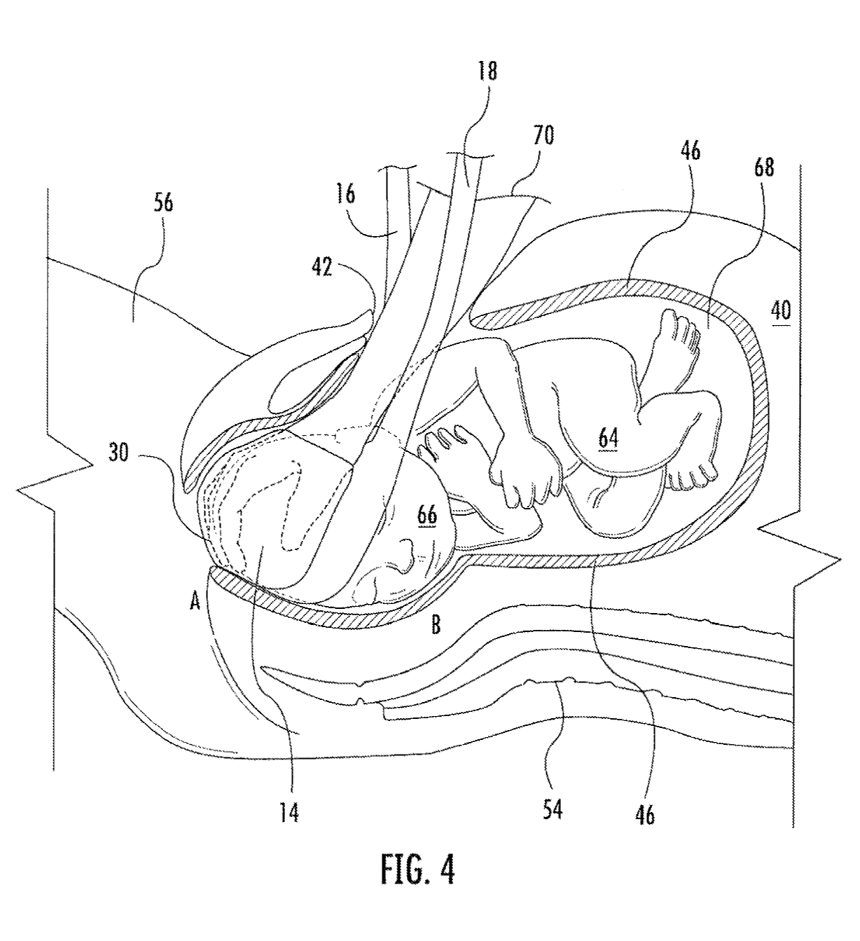

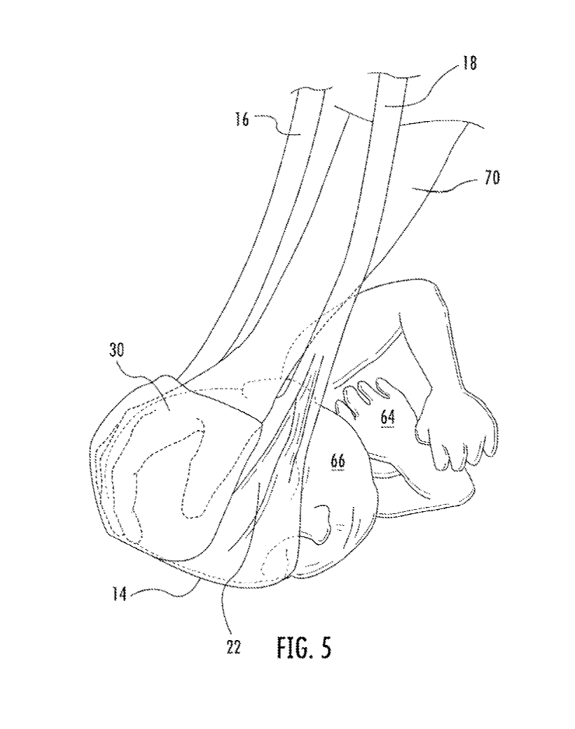

FIG. 4 illustrates the surgeon's hand 30, which is within pocket 24, inserting article 10 through the uterine incision 42 and under the fetal head 66 of the fetus 64. The surgeon's arm 70 can be seen extending into the uterus 68 of the woman 40. FIG. 4 illustrates a typical position for the obstetrician's hand 30 to be located in order to start the delivery of the fetus 64 during a cesarean section delivery procedure.

FIG. 5 illustrates a more detailed illustration of the center portion 14 when the center portion 14 is expanded around fetal head 66 of the fetus 64 during a cesarean section procedure. FIG. 5 illustrates how pleats 20 and 22 allow for the expansion of the center portion 14 around the head 66 of the fetus 64 and how narrow end portions 16 and 18 are configured to extend outside of the uterine incision 42 and the woman 40. The surgeon's hand 30 and the surgeon's arm 70 are illustrated in their positions in preparation to move the head 66 of the fetus 64. As illustrated in FIG. 5, the center portion 14 may be configured to fit snugly and gently around the fetal head 66 of the fetus 64. As shown in FIG. 5, by pulling on end portions 16 and 18 of the planar member 12, additional force may be added to surgeon's hand 30 and may allow the surgeon's hand 30 to more gently guide the fetal head 66 of the fetus 64 during the delivery of the fetus 64.

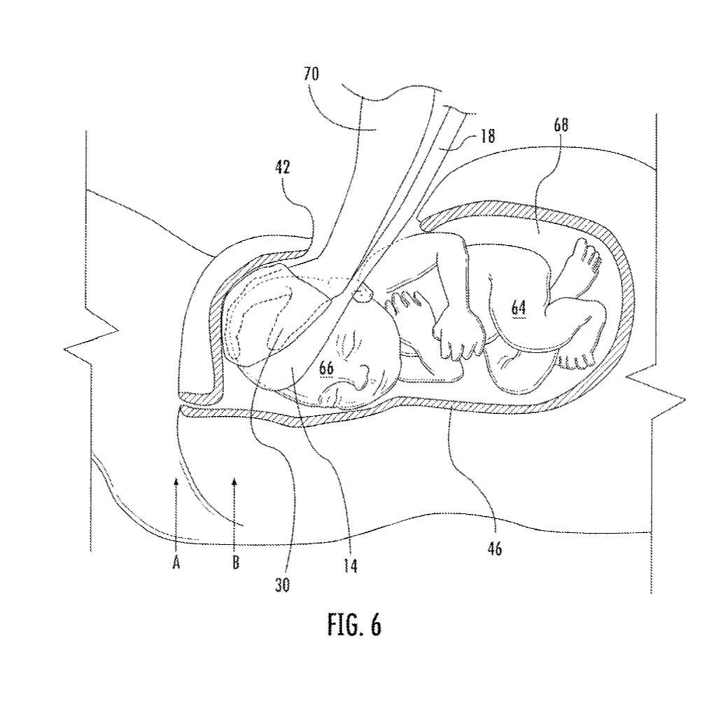

FIG. 6 illustrates the position of the fetal head 66 of the fetus 64 after the surgeon's hand 30 has be used to guide fetal head 66 of the fetus 64 from Location A to Location B. At location B, the surgeon is now able to flex the fetal head 66 of the fetus 64 and deliver the fetal head 66 and fetus 64 through the uterine incision 42 that the surgeon made on the pregnant woman 40. At this point in the delivery process, further lifting on end portions 16 and 18 may not be needed and the surgeon's hand 30 should be able to deliver fetus 64 through the uterine incision 42 in a routine manner.

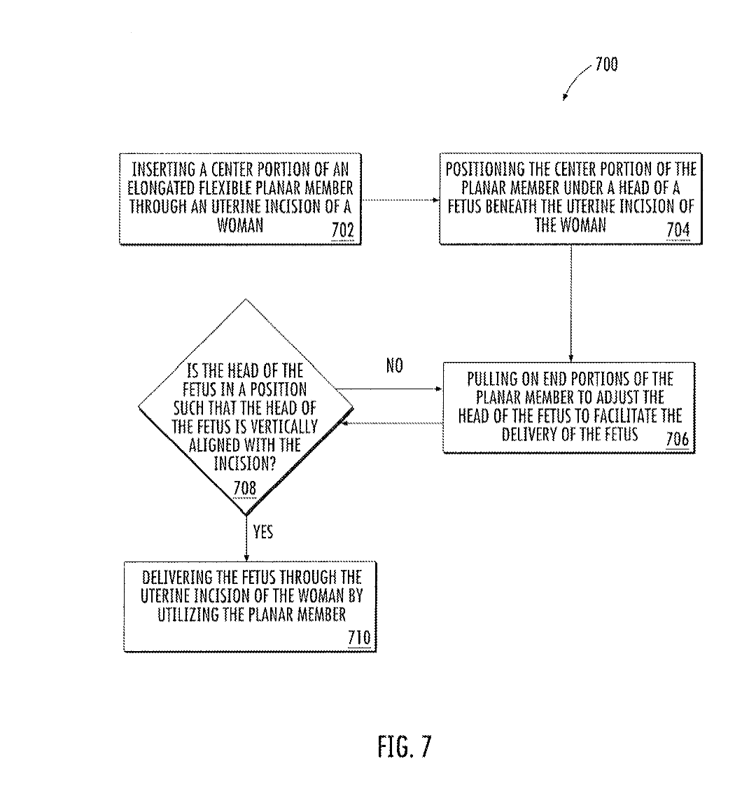

As shown in FIG. 7, an exemplary method 700 for assisting a cesarean section deliver is schematically illustrated. The method 700 may include, at step 702, inserting a center portion 14 of an elongated, flexible planar member 12 of an article 10 through an uterine incision 42 of a woman 42, such as during a cesarean section delivery procedure or other procedure utilized for delivering a fetus 64. At step 704, the method 700 may include positioning the center portion 14 of the planar member 14 under a head 66 of the fetus 64 to be delivered. In one embodiment, the method 700 may include having the surgeon insert the center portion 14 of the planar member 12 by placing his or her hand 30 into a pocket 24 of the planar member 12 and then guiding the center portion 14 of the planar member 12 under the head 66 of the fetus 64. Once the center portion 14 of the planar member 12 is positioned securely under the head 66 of the fetus 64, the method 700 may include pulling on the end portions 16 and 18 of the planar member 12 to adjust the head 66 of the fetus 64 to facilitate the delivery of the fetus 64 at step 706. In one embodiment, by pulling on the end portions 16 and 18 of the planar member 12, additional lift force may be provided to assist in adjusting the head 66 of the fetus 64 and delivering the fetus 64 via the uterine incision 42.

At step 708, he method 700 may include determining if the head 66 of the fetus 64 is in a position such that the head 66 of the fetus 64 is vertically aligned with the uterine incision 42. In one embodiment, the method 700 may include determining if any other body part of the fetus 64 is in a position such that the other body part of the fetus 64 is aligned with the uterine incision 42 or is aligned any other manner that is suitable for delivering the fetus 64 safely from the woman 40. If the head 66 of the fetus 64 is determined to be in a position such that the head 66 of the fetus 64 is not vertically aligned with the uterine incision 42, the method 700 may include continuing step 706 by continuing to pull on the end portions 16 and 18 of the planar member 12 to further adjust the head 66 of the fetus 64 to facilitate the delivery of the fetus 64. Once the head 66 of the fetus 64 is in a position that is vertically aligned with the uterine incision 42, the method 700 may include delivering the fetus 64 through the uterine incision 42 of the woman 42 at step 710. In one embodiment, the method 700 may include utilizing the planar member 12 of the article 10 to assist in providing lift force to deliver the fetus 64 via the uterine incision 42.

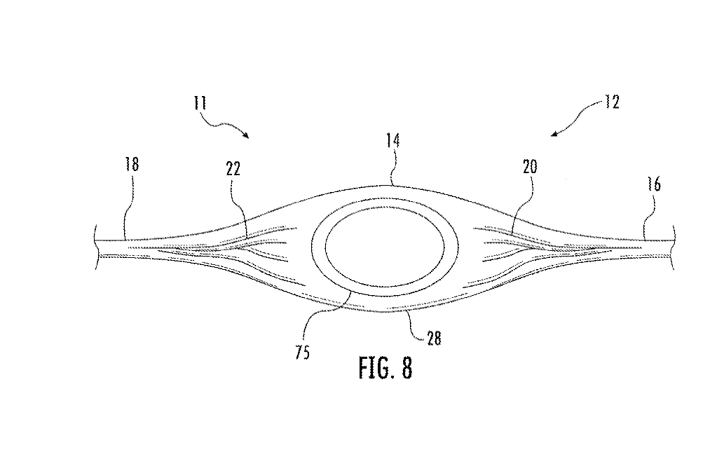

In one embodiment, the article 10 and methods described herein may incorporate further features that may be utilized to assist in the delivery of the fetus 64, such as during a cesarean section delivery procedure or other similar procedure. Referring to FIG. 8, an article 11 for assisting with a delivery of a fetus 64 during a cesarean section delivery procedure is disclosed. Notably, article 11 may include any and all of the components of article 10, however, article 11 may further include a suction device 75. The suction device 75 may be positioned on the center portion 14 (fetal head surface) of the article 10 or at any other desired location on the article 11. In one embodiment, when the planar member 12 of the article 11 is positioned under the head 66 or other body part of the fetus 64, the suction device 75 may be positioned on the underside of the head 66 or other desired body part of the fetus 64. Once the planar member 12 of the article 11 is lifted to an appropriate level and with adequate space, the suction device 75 may be pumped to a safe pressure by utilizing a vacuum system or other device that may be utilized to provide a suction force to the suction device 75 so that the suction device 75 can gently hold onto the head 66 or other desired body part of the fetus 64. In one embodiment, the vacuum system may be similar to vacuum devices that are traditionally available.

Once the safe pressure is achieved for the suction device 75, the suction device 75 may provide a gentle hold on the leading point of the fetal head 66, which would allow the delivering obstetrician to deftly guide the head 66 out of the uterine and abdominal incisions 42. Notably, this process may be performed without needing the obstetrician's hand being inserted into the uterine incision 42 and behind the head 66 of the fetus 64. As a result, for surgeons with large hands, this may provide for substantial space savings. In addition, the gentle hold provided by the suction device 75 may reduce the chance of the now freed head 66 of the fetus 64 from rising up too high and flipping towards the side, which can cause the head 66 to be positioned in a transverse or oblique position. If the head 66 is in a transverse or oblique position, it may lead to further delays in the cesarean section procedure and may cause further difficulties in maneuvering the fetus 64, which may increase the potential for trauma.

In another embodiment, the planar member 12 of the article 10 may envelope the surgeon's delivery hand 30, which often times may be further double-gloved for the cesarean section procedure. Typically, time is rarely taken to change gloves during the cesarean section procedure because maximal bleeding may occur after delivery and up until the closure of the uterus 68. The surgeon's operating glove may remain more sterile than it would have been if he or she was not using article 10 or 11. In one embodiment, the article 10 or article 11 may even be removed vaginally rather than up through the uterine incision 42 after its role of lifting the head 66 is performed. As a result, this may further isolate the more sterile operating area from the bacterial flora of the vaginal area 50. Of course, routine antibiotic prophylaxis may still be utilized in these cases and accepted as clean and/or contaminated by operating room standards, but a further prophylactic benefit from post-operative maternal infection is certainly possible.

In one embodiment, during clinical use, the placement of the article 10 or 11 may be greatly facilitated by a vaginal assistant, especially in extreme cases of impaction. While this may necessitate time consuming positioning and draping of the patient for the procedure beforehand (one of the drawbacks of current methods of vaginally assistance), it would allow the article 10 or 11 to be placed precisely with no need to fully interpose the entire hand 30 of the surgeon between the head 66 of the fetus 64 and maternal tissues of the woman 40. The surgeon may gently feed the article 10 or 11's leading edge 28 down to where the vaginal assistant can gently spread the article 10 or 11 under and beyond the leading edge of the head 66. Then, upwardly lifting the end portions 16 and 18 of the article 10 or 11 will perfectly snug the back edge of the article 10 or 11 into place behind the head 66. As a result, rather than having localized and potentially harmful pressure of a vaginal lift (especially if the vaginal assistant is inexperienced), the article 10 or 11 will naturally spread out the lifting pressure and yet provide full lifting power for the obstetrician.

In one embodiment, the article 10 or 11 may be utilized to assist in reducing the incidence of uterine trauma, bladder trauma, maternal bleeding, blood transfusion, infection, and delays in delivering the fetus 64. The article 10 or 11 may be utilized in situations where the head 66 of the fetus 64 is rapidly progressed downward, while preparing for delivery or the uterine incision 42 cannot be as large as desired for the size of the fetal head 66.

As described herein, article 10 and article 11 of the present disclosure may be used as an aid to the obstetrician's delivering hand 30 in the process of dislodging and elevating the deeply engaged fetal head 66, elevating it to a level of the uterine incision 2, where it can be flexed and delivery can occur by following normal routine procedures. Article 10 and article 11 of the present disclosure overcomes the great resistance that the fetal head 66 has at a point where it is jammed in place between bones and soft tissues of the maternal pelvis.

Additionally, as will be appreciated by those of skill in the art, the location of uterine incision 42 may be done in order to minimize the lifting of the fetus 64 through the uterine incision 42, thus, it must be low enough to reach the tip of the head 66, yet high enough in the uterus 68 to not to be in the vagina itself.

Sterile lubricant or saline can be used in the process of the present disclosure in order to improve the sliding ability of the article 10 or 11 between the head 66 of the fetus 64 and the uterus 68. Also, the intrinsic nature of the fabric of the article 10 or 11 may be such that it minimizes friction.

The pocket 24 can be either an open pocket, similar to a shirt pocket, or it can be designed to be like a glove with individual finger pockets in order to provide the surgeon with increased control over the center portion of the article 10 or 11 when guiding it underneath the head 66 of the fetus 64.

The illustrations of arrangements described herein are intended to provide a general understanding of the structure of various embodiments, and they are not intended to serve as a complete description of all the elements and features of the article and methods that might make use of the structures described herein. Many other arrangements will be apparent to those of skill in the art upon reviewing the above description. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

What is claimed is:

1. An article for assisting a cesarean section delivery, comprising:
an elongated, flexible planar member; and
a pocket centrally located on the planar member, wherein the pocket is adapted to fit a hand, and wherein the pocket comprises an opening for inserting the hand into the pocket of the article.

2. The article of claim 1, wherein the planar member comprises two sets of pleats longitudinally oriented on the planar member, and wherein the pocket is positioned between the two sets of pleats.

3. The article of claim 1, wherein the planar member has a center portion in which the pocket is located and further comprises end portions.

4. The article of claim 1, wherein the article is made of a synthetic fabric that withstands sterilization.

5. A method for cesarean section delivery, comprising:
inserting a center portion of an elongated, flexible planar member through an uterine incision, wherein end portions of the planar member remain outside of the uterine incision;
placing the center portion of the planar member under a head of a fetus; and
pulling on the end portions of the planar member to move the head of the fetus and facilitate delivery of the fetus through the uterine incision.

6. The method of claim 5, wherein the planar member has a pocket located in the center portion and the pocket has an opening, and wherein a hand of a delivering person is inserted into the pocket through the opening, and wherein the hand guides the center portion of the planar member under the head of the fetus during placement of the center portion under the head of the fetus.

7. The method of claim 6, wherein after pulling, lifting the head of the fetus through the uterine incision based in part on adjusting the planar member.

8. The method of claim 5, wherein pulling on the end portions is stopped once the head of the fetus is aligned vertically with the uterine incision.

9. A method for cesarean section delivery, comprising:
inserting a center portion of an elongated, flexible planar member through a uterine incision, wherein end portions of the planar member remain outside of the uterine incision;
placing the center portion of the planar member under the head of a fetus; and
delivering the fetus through the uterine incision, wherein the center portion of the planar member is under the head of the fetus.

10. The method of claim 9, further comprising pulling the end portions of the planar member to facilitate delivery of the fetus.

11. The method of claim 9, wherein the planar member has a pocket located in the center portion and the pocket has an opening, wherein a hand of a delivering person is inserted into the pocket and the hand guides the center portion of the planar member under the head of fetus during placement of the center portion under the head of the fetus.

12. The method of claim 11, further comprising pulling the end portions of the planar member to facilitate delivery of the fetus during the cesarean section delivery.

13. The method of claim 12, further comprising lifting the head of the fetus through the uterine incision after pulling the end portions.

14. The method of claim 13, further comprising ceasing pulling the end portions of the planar member once the head of the fetus is aligned vertically with the uterine incision.

15. The method of claim 9, further comprising activating a suction device of the planar member to suction onto the head of the fetus after placing the center portion of the planar member under the head of the fetus.

16. The method of claim 9, further comprising expanding the center portion of the planar member by utilizing a plurality of pleats positioned on the planar member.

17. The method of claim 9, further comprising creating the uterine incision prior to inserting the center portion of the elongated, flexible planar member through the uterine incision.

18. The method of claim 9, further comprising placing the center portion of the planar member under a body part other than the head of the fetus.

19. The method of claim 18, further comprising lifting the body part of the fetus through the uterine incision after pulling the end portions.

20. The method of claim 9, wherein the head of the fetus is impacted in a vaginal area.
